(19) United States
(12) Patent Application Publication
(10) Pub. No.: US 2006/0106406 A1
(43) Pub. Date: May 18, 2006

(54) Methods and devices for extravascular intervention

(76) Inventor: Judah Weinberger, Teaneck, NJ (US)

Correspondence Address: Leslie Gladstone Restaino;Brown Raysman Millstein Felder & Steiner LLP, 163 Madison Avenue, P.O. Box 1989, Morristown, NJ, 07962-1989, US

(21) Appl. No.: 10/992,297

(22) Filed: Nov. 18, 2004

(51) Int. Cl. A61B 17/08

(52) U.S. Cl. 606/153

(57) ABSTRACT

The present invention relates to methods for treating an aneurysm or other vascular condition in a patient by implanting an extravascular intervention device to partially encircle a blood vessel, or portion thereof, having the vascular condition. The present invention also provides extravascular devices for use in treating patients with aneurysms and other vascular conditions. The extravascular intervention devices provide extravascular support to vascular walls of an aneurysm, thereby reducing the stress on the aneurysm walls and reducing the risk of rupture. The extravascular intervention devices may further be used as means for administering therapeutic agents to vascular targets extravascularly.

### RELATED APPLICATION

This application is a continuation-in-part application of U.S. application Ser. No. ______ (not yet assigned), entitled METHODS AND DEVICES FOR EXTRAVASCULAR INTERVENTION, which was filed on Sep. 27, 2004.

### BACKGROUND OF THE INVENTION

The invention generally relates to methods and devices for vascular intervention. Particularly, the present invention provides methods and devices for treating vascular diseases or conditions, including, but not limited to, aneurysms. An aneurysm is generally a dilation, widening, or bulge of a weakened wall of a blood vessel. Although aortic aneurysms are the most common types of aneurysm, aneurysms may occur in any artery. It is understood, therefore, that although the present invention will be described by way of example in relation to the treatment of aortic aneurysms, the methods and devices described herein are not limited to the treatment of aortic aneurysms, and may be applied to the treatment of other types of aneurysms and other vascular conditions.

Aneurysms are typically associated with, or secondary to, arteriosclerosis, trauma to the aorta, inflammation of the wall of the aorta, and hereditary conditions such as Marfan's syndrome. A patient afflicted with an aneurysm may experience pain and discomfort, particularly as the aneurysm grows. Additionally, aneurysms may rupture, posing a relatively high mortality risk that is estimated at about 50% for abdominal aortic aneurysms and greater for thoracic aneurysms. The risk associated with rupture is generally based on the size of the aneurysm in relation to the normal or average size for the particular vessel afflicted with the aneurysm. For example, the risk of rupture within two years of discovery is about 50% for an aortic aneurysm measuring 5-6 cm, or greater.

Presently, aortic aneurysm treatments or interventions are generally limited either to replacement of the affected section of the aorta with a prosthetic graft, or implantation of an endovascular graft or endograft within the aorta. However, replacement with a prosthetic graft requires invasive and complex open-surgical intervention, with an estimated full recovery of about 6 weeks thereafter. Furthermore, open-surgical intervention is associated with a relatively high risk of mortality: about 5% for abdominal aortic aneurysms and about 15% for thoracic abdominal aneurysms. The high risk of mortality associated with open-surgical intervention has generally limited the procedure to the treatment of aortic aneurysms of about 5-6 cm or greater.

Endovascular graft implants are typically fabric-covered metallic stents that are passed in a collapsed orientation, through a femoral artery, into the dilated section of the aorta. At the aorta, the stent graft is expanded to secure the endovascular graft to normal or unaffected sections of the aorta, above and below the aneurysmal section of the aorta. Although less invasive than a surgical prosthetic graft, endovascular graft implants are limited to use in patients having anatomy suited for endovascular grafts. For example, since the endovascular graft typically passes through the femoral artery, patients having narrow and/or occluded femoral or iliac arteries may not be suited for treatment with an endovascular graft. Additionally, endovascular grafts, and the devices necessary for their implantation, are relatively complex, resulting in a costly procedure. They also have a tendency to migrate after implantation or develop leaks.

Navigational requirements for the endovascular graft implant further prevent its use in the treatment of aneurysms in vessels with acute bends, such as the arch of the aorta, and in vessels through which one must navigate to reach the target vessel, such as the ascending aorta. The risk of mortality associated with the endovascular graft is estimated to be about the same as that of the open-surgical intervention procedure; thus, the use of endovascular graft implants is also limited to the treatment of aneurysms of about 5-6 cm or greater.

There is, therefore, a need for methods and devices for aneurysm intervention that entail a lesser degree of complexity in their performance, and a lower cost associated therewith. There is also a need for methods and devices for aneurysm intervention that exhibit a lower rate of mortality and, as such, are suitable for the treatment of patients independent of the size of the aneurysm, and, particularly, are suitable for the treatment of aneurysms of less than 5 cm. There is a further need for methods and devices for preventing endovascular graft implant migration. Finally, there is also a need for methods and devices generally suitable for the treatment of a patient with an aneurysm, independent of the patient's endovascular anatomy.

### SUMMARY OF THE INVENTION

In one aspect of the invention, a method is provided for treating a patient having a vascular condition, such as an aneurysm, that includes the steps of introducing an extravascular device to a site of interest in the patient, to access the exterior of a blood vessel having a section affected by the vascular condition, and implanting the extravascular device to partially encircle at least a portion of the section of the blood vessel affected by the vascular condition, thereby providing extravascular support to the affected section of the blood vessel. The extravascular device, when implanted, generally has a size that is suitable for either maintaining the affected size of the affected section or displacing vascular walls of the affected section inwardly.

In one embodiment, the affected section of the blood vessel comprises an aneurysm, and the extravascular device has a structure that is capable of being formed into a tubular shape that, upon implantation of the device, forms a tubular structure having an inner surface that contacts the exterior of the aneurysm's vascular walls, thereby providing compressive forces thereto for supporting the vascular walls of the aneurysm. The device may further include at least one access opening that allows the affected blood vessel to be placed through the tubular structure. In one embodiment, the access opening comprises a pair of longitudinal ends that are connected to each other, thereby closing the tubular structure for support of the affected blood vessel.

In one embodiment, the device is made of a suitable biocompatible and MRI-compatible material which is resilient against arterial blood flow pressure, biocompatible and preferably MRI compatible material resilient against arterial blood flow pressure and has a preformed tubular shape that is resumed when the device is implanted to partially encircle the affected section of the blood vessel. The device may further include a coating comprising at least one therapeutic agent, e.g., based on systemic relevance, on at least a portion or all of the device's inner surface and/or the device's outer surface.

In one embodiment, the device is a collapsible extravascular intervention device that includes a plurality of longitudinal members connected to each other by a plurality of flexural members, each flexural member having a bend that, upon application of force, provides flexure resulting in a narrowing of lateral distances between the longitudinal members. The device may further include two end longitudinal members and at least one central longitudinal member. In this instance, each of the longitudinal members has a first and second end that are connected by flexural members to an adjacent longitudinal member's first end and second end, respectively. The two end longitudinal members may also have corresponding interlocking geometries.

In one embodiment, the collapsible device is made of a biocompatible and preferably MRI compatible material resilient against arterial blood flow pressure and has a preformed tubular shape and a preformed expanded shape that are resumed when the device is implanted to partially encircle the affected section of the blood vessel to form a tubular structure.

In another aspect of the invention, an extravascular device is provided for use in treating a blood vessel affected by a vascular condition, wherein the device includes a structure capable of being formed into a tubular shape that, upon implantation of the device to partially encircle the affected section of the blood vessel, forms a tubular structure that has an access opening that allows the affected blood vessel to be placed through the tubular structure. The device, when implanted, has a size, e.g., a diameter, for either maintaining the affected size of the affected section of the blood vessel or displacing the vascular walls of the affected section of the blood vessel inwardly.

In one embodiment, the device includes a plurality of longitudinal members connected to each other by a plurality of flexural members, thereby forming the tubular structure. Each flexural member is capable of bending upon application of force so that the lateral distances between the longitudinal members are narrowed, thereby providing collapsibility to the device. In another embodiment, the device includes two end longitudinal members and at least one central longitudinal member. Each of the longitudinal members has a first end and a second end connected by flexural members to an adjacent longitudinal member's first end and second end, respectively. The two end longitudinal members may also include corresponding interlocking geometries.

In one embodiment, each flexural member includes at least one semicircular flexible element, such as a semicircular flexible element defined by obtuse circumferential geometry and acute circumferential geometry. In this instance, the obtuse circumferential geometry comprises a pair of first central arcs with centers separated by a first central distance and a pair of first exterior arcs each tangential to one of the first central arcs and the flexural members, and the acute circumferential geometry comprises a pair of second central arcs with centers separated by a second central distance and a pair of second exterior arcs intersecting tangentially with the second central arcs and intersecting non-tangentially with the flexural members.

In one embodiment, the tubular structure includes means for administering a therapeutic agent extravascularly, such as a coating on at least a portion of the inner surface of the device that includes at least one therapeutic agent.

In another aspect of the invention, an extravascular intervention device is provided that has a structure made of a biocompatible and preferably MRI compatible material resilient against arterial blood flow pressure, wherein the structure is capable of being formed into a tubular shape that, upon implantation of the device to partially encircle a blood vessel, forms a collapsible tubular structure having an access opening that allows a blood vessel to be placed through the tubular structure. The structure has a preformed tubular shape and a preformed expanded shape that are resumed when the device is implanted to partially encircle the blood vessel, thereby allowing the blood vessel to be placed through the collapsible tubular structure. In one embodiment, the preformed tubular and expanded shapes provide at least a minimal amount of compressive force on the blood vessel when the device is implanted to partially encircle the blood vessel to provide extravascular support thereto. In another embodiment, the collapsible tubular structure has an inner surface that includes deployable barbs and/or a roughened surface.

In another aspect of the invention, an extravascular intervention device is provided that includes a structure capable of being formed into a tubular shape that, upon implantation of the device to partially encircle a blood vessel, forms a tubular structure having an inner surface and an access opening that allows the blood vessel to be placed through the tubular structure, and means for administering a therapeutic agent to the blood vessel extravascularly, such as a coating including the therapeutic agent that is placed on at least a portion of the inner surface of the tubular structure so that the therapeutic agent comes into contact with the exterior of the blood vessel.

The extravascular device of the present invention avoids the need for intravascular surgery such as a thoractomy. The device may be place via thoracoscopic surgery or VATS (video-assisted thoracic surgery) whereby small incisions are made in the chest. The thorascope guides the placement of the extravascular device.

Additional aspects of the present invention will be apparent in view of the description which follows.

### BRIEF DESCRIPTION OF THE FIGURES

FIG. 1 is an anterior plan view of a human aorta, showing typical locations whereon aortic aneurysms may occur;

FIG. 2 is a cross-sectional view of a blood vessel having an aneurysm;

FIGS. 3a-3b are plan views of extravascular intervention devices disposed around a vessel having an aneurysm;

FIGS. 4a-4f are perspective views of several embodiments of extravascular intervention devices;

FIGS. 5a and 5b are plan views of several embodiments of extravascular intervention devices disposed around a human aorta at typical locations whereon aortic aneurysms may occur;

FIGS. 6a-6d are views of an extravascular intervention device according to one embodiment of this invention; and

FIG. 7 is a plan view of extravascular intervention devices further implanted around an aneurysm with mesh or fabric between the struts.

### DETAILED DESCRIPTION OF THE INVENTION

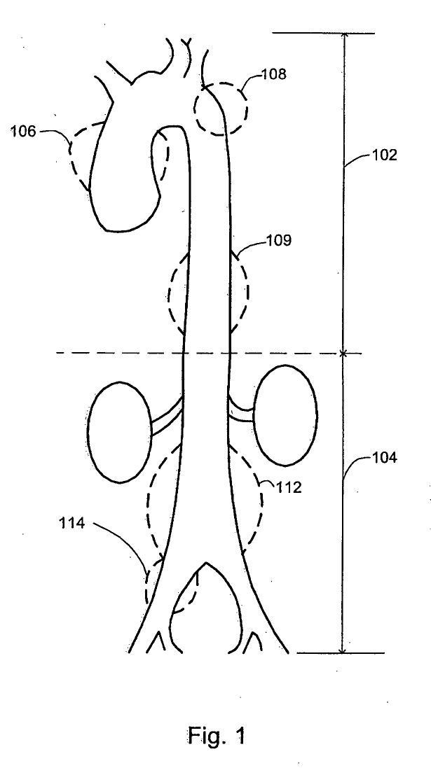

Aneurysms may occur in any blood vessel; however, aneurysms commonly and typically occur in the aorta. An anterior view of a human aorta shown in FIG. 1 depicts typical locations on the aorta where aneurysms may occur. Referring to FIG. 1, aortic aneurysms may occur in the thoracic cavity 102 or in the abdominal cavity 104. Aneurysms in the thoracic cavity may occur in any location thereon, such as on the ascending aorta 106, the arch of the aorta 108, the descending aorta 109, the thoracic aorta 110, or any vessels branching therefrom. Aneurysms in the abdominal cavity may similarly occur in any location thereon, such as on the abdominal aorta 112, the iliac arteries 114, or any vessels branching therefrom.

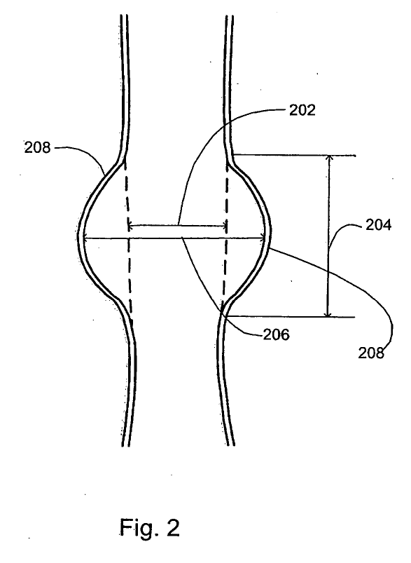

Aneurysms generally occur in vessels having weakened walls. Blood pressure in such vessels causes dilating or bulging at the weakened vessel walls, and, ultimately, the weakened walls may be overcome by the blood pressure. Referring to FIG. 2, a vessel having a normal size or diameter 202 may bulge or dilate at a section of the vessel having a wall weakened by a vascular disease; this weakened wall may then result in an aneurysm. An aneurysm is typically referred to by size or by aneurysm diameter 206. Since aneurysms are typically asymmetric and non-concentric, an aneurysm size, e.g., diameter 206, is used herein generally to refer to a dimensional distance between the vessel walls 208 affected by the vascular condition. The average normal diameter 202 of an adult human aorta, for example, ranges, on average, between 2 cm and 2.5 cm. Aneurysm diameters 206 may vary from just above the normal diameter to a size reportedly as high as 21 cm. Blood pressure acting on the affected vessel walls further causes the aneurysm to dilate or grow until either rupture, dissection, or treatment/intervention.

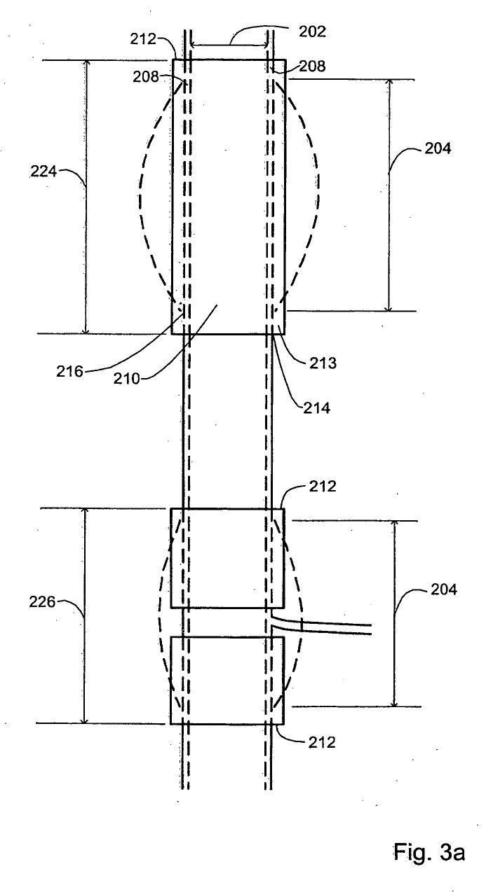

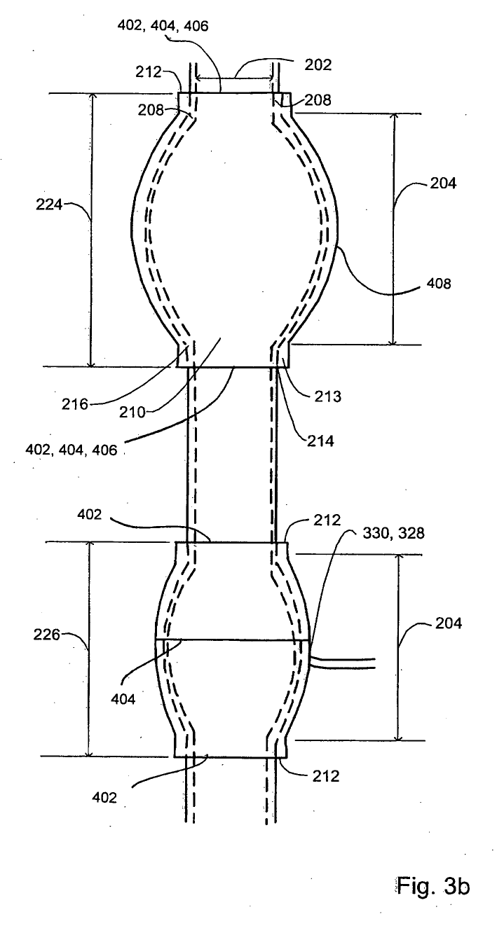

Referring to FIGS. 3a and 3b, a device useful for extravascular intervention, i.e., an extravascular intervention device 212 according to the present invention, is generally a device capable of providing extravascular support to weakened or otherwise affected vessel walls 208. Where the affected blood vessel has an aneurysm, the extravascular intervention device of the invention reduces or eliminates the rate of dilation of the aneurysm and/or reduces the risk of rupture. The functionality described herein may be achieved with extravascular intervention devices configured or produced in a variety of ways, and, therefore, is not limited to the examples provided herein. Extravascular support may be provided, for instance, with a tubular extravascular intervention device 212 that is capable of being placed to partially encircle an affected blood vessel 210, e.g., a blood vessel having an aneurysm 210, or a portion thereof, to provide extravascular support for, and to relieve the stress on, the affected vessel walls 208. Hereinafter the term “around” is used synonymous with the term “to partially encircle.”

In one embodiment, the extravascular intervention device 212 is dimensionally configured or produced such that, when implanted around a blood vessel with an aneurysm 210, the aneurysm walls 208 are maintained at a desired size, as shown in FIG. 3b, and/or are inwardly displaced in relation to the central axis of vessel, as shown in FIG. 3a. The affected vascular walls may be maintained by dimensionally configuring the device 212 to have a size that, when implanted around the vessel or aneurysm walls 208, provides extravascular support thereto and retains the affected section in a desired size—such as the affected shape and size, e.g., dilated shape and size of the affected section—thereby preventing further dilation and/or rupture.

Alternatively, or in addition, the affected vascular walls may be displaced inwardly with the device 212, in order to manipulate the vessel with the aneurysm 210 into a desired size, such as the size of the unaffected vessels flanking the aneurysm, e.g., the normal vessel size for the particular vessel, or any other shape and size. For example, the walls of an aortic aneurysm having a 6-cm aneurysm diameter 206 may be maintained at 6 cm, or displaced so as to manipulate the vessel into a cylindrical shape having a diameter approximately equal to either a normal diameter of about 2-2.5 cm, or a diameter between about 2 cm and about 6 cm. In certain instances, displacement of the vascular walls, to significantly reduce the size of the aneurysm, may cause the aneurysm walls to fold, thereby disrupting the generally circular cross-sectional geometry of the blood vessel. Accordingly, in one embodiment, the affected vascular walls are displaced inwardly with the device 212, in order to provide extravascular support thereto, e.g., without creating folds in the displaced vascular walls. Additionally, removal of transmural stresses may cause the diameter of the aneurysm to shrink or get smaller, with or without administering therapeutic agents in this respect.

In one embodiment, the extravascular intervention device 212 is generally a structure capable of being formed into a tubular shape that, upon implantation of the device around an aneurysm, forms a tubular structure 213 having one or more inner surfaces 214 that come into contact with the exterior side of the aneurysm walls 216 and compress, or provide compressive forces for supporting, the aneurysm walls 208. The compressive forces act on the aneurysm walls 208 to maintain a desired size and/or to displace them inwardly to produce a desired size. The longitudinal length of the device 224 may be less than, equal to, or greater than the longitudinal length of the aneurysm 204. “Longitudinal” is herein used as a directional reference that is in line with the central axis of the vessel having the aneurysm. The longitudinal length of the device 224 is preferably equal to, or in excess of, the longitudinal length of the aneurysm 204, thereby providing support over at least the longitudinal length of the aneurysm 204.

Support for the aneurysm walls may also be provided with a plurality of extravascular intervention devices 212 implanted in a stacked arrangement to yield an effective longitudinal length 226 that may be less than, equal to, or greater than the longitudinal length of the aneurysm 204. A plurality of extravascular intervention devices 212, for example, may be used on aneurysms that have vessels branching therefrom, in order to accommodate the branching vessels and provide support over an effective longitudinal length 226 equal to or greater than the longitudinal length of the aneurysm 204. Referring to FIG. 3b, a plurality of extravascular devices 212 may be stacked in an overlapping arrangement, in order to provide continuous support over the effective longitudinal length 204 while accommodating the branching vessels. By way of example, at least one of the devices may have an aperture 328, 330 to accommodate branch vessels. Dilated branch vessels may similarly be supported with a sleeve exiting from the aperture 328, 330. The extravascular intervention devices may also be locked together to prevent or limit migration after implantation. The locking mechanism may be integrated into the device itself, such as with complimentary geometry for connecting or locking the ends of the devices together in a stacked arrangement, such as pegs that fasten to recesses, hooks that fasten to holes, buttons, snaps, etc., or may be provided separately, e.g., a plurality of devices may be sutured together longitudinally.

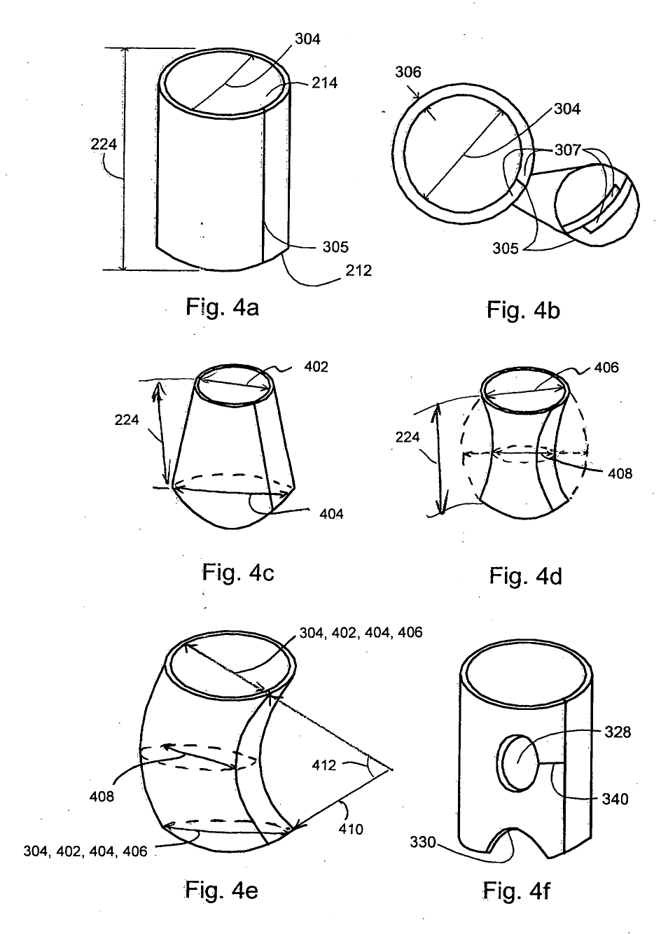

As shown in FIGS. 4a-4e, the extravascular intervention devices 212 may be configured or produced in a variety of shapes and sizes, correspondingly to enable retention of the vessel with the aneurysm 210 or manipulation of the vessel into various shapes and sizes. For example, the extravascular intervention device 212 may be configured or produced such that its inner surfaces 214 provide compressive forces to enable manipulation of the vessel with the aneurysm 210 into cylindrical shapes, conical shapes, hour-glass shapes, spherical segment shapes, curved cylindrical shapes, etc. Referring to FIGS. 4a-b, a cylindrical extravascular intervention device has a longitudinal length 224, a thickness 306, and an inner diameter 304. The longitudinal length 224, thickness 306, and inner diameter 304 may be of any combination to accommodate various blood vessels and various aneurysm lengths, such as lengths of about 1 mm to about 30 cm, and inner diameters of about 3 mm to 21 cm. Lengths between about 1 cm to about 3 cm may beneficially be implanted with minimally invasive surgical procedures. Longer lengths between about 3 cm to about 12 cm or greater may require open visual interventions. The devices may also have rounded or flared ends to protect the tissue at the site of the implantation against abrasion or tearing therewith.

In one embodiment, extravascular intervention devices 212 include at least one access opening 305 to facilitate implantation thereof around vessels having aneurysms 210. The access opening 305 is a lengthwise discontinuity in the generally tubular structure of the device, which allows vessels to be placed therethrough. Although the access opening 305 is shown as a straight line, it is understood that the geometry of the opening may be any one or more of a variety of non-linear shapes, including circular, parabolic, elliptical, etc. The compressive forces necessary to maintain or manipulate an aneurysm in a desired shape and size is attained by fastening or otherwise connecting the longitudinal ends 307 of the extravascular intervention device 212 that represent the access opening when placed around the vessel having the aneurysm 210, such that the ends remain essentially fixed in relation to each other.

The ends may be fastened in a variety of ways, depending on the materials and construction of the device 212. For instance, where the device is of a flexible construction, such as a fabric sheet, elastic or otherwise, and formed into a tubular structure, the longitudinal ends 307 may be fastened to each other by sutures, self closing, e.g., dynamic, or otherwise, wires, staples, clamps, ties, pins, Velcro, zipper(s), buttons, snaps, hooks, or any type of tension mechanism, glue/bonding agents, magnets, welding, e.g., with laser, etc., or by any other type of means for fastening the longitudinal ends 307. Where the device is constructed of a less flexible or essentially rigid material, the longitudinal ends 307 may additionally be fixed in relation to each other with resistance provided by an essentially rigid construction. Alternatively, or in addition, the extravascular intervention device 212 may include fastening means disposed thereon such that the longitudinal ends 307 engage each other, as with an arrangement similar to tie wraps, in order to provide a device with a variable diameter. The longitudinal ends 307 may be fastened such that the ends butt or overlap against each other, as shown in FIG. 4b.

Referring to FIG. 4c, the extravascular intervention device 212 may be configured or preformed to have a general or right conical shape, circular or otherwise, or portions thereof. The conical shape is generally characterized with having a minor diameter 402 and a major diameter 404 and a length 224. Referring to FIG. 4d, the extravascular intervention device 212 may be also be configured to have a shape in the form of an hour-glass or a spherical segment, circular or otherwise, or portions thereof, which is characterized with having an end diameter or diameters 406 and a central diameter 408, which is less than the end diameter 406 for the hour-glass shape and greater than the end diameter 406 for the spherical segment shape.

Each one of the tubular shapes, e.g., the cylindrical, conical, hour-glass, and spherical shapes, may be configures to assume a curved shape as shown in FIG. 4e, which shows the curved cylindrical shape. The curved shape is characterized as having a curve radius 410, an angular measurement 412, and diameters 304 for a curved cylinder, diameters 404 and 404 for a curved conical shape, and diameters 406 and 408 for a curved hour-glass or spherical segment shapes. The curve radius 410 and the angular measurement 410 may vary to accommodate or resemble the curvature of the affected blood vessel, such as in the ascending aorta. The angular measurement 410 may vary between about greater than one degree and about 360 degrees, or greater, or more preferably between about 180 degrees to nearly about 360 degrees to, e.g., accommodate the ascending aorta and the aortic arch.

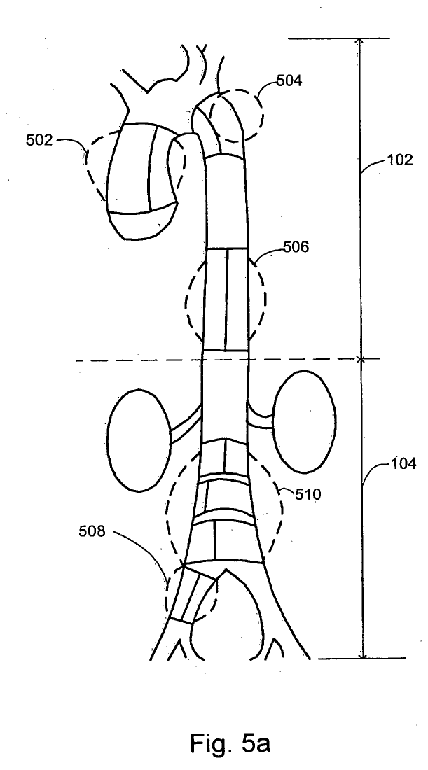

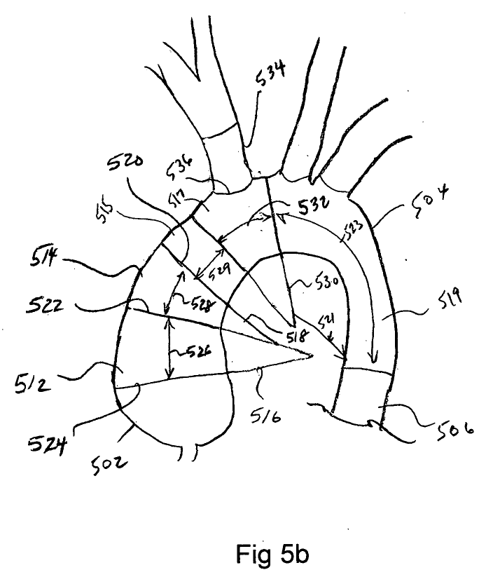

Referring to FIG. 4f, the extravascular intervention device 212 may further include one or more apertures 328, 330 to facilitate implantation around vessels having aneurysms that occur in locations with vessels branching therefrom. For example, the extravascular intervention device 212 may include an aperture communicating with circumferential ends of the tubular structure 330 and/or a central aperture 328 having an access opening 340 to accommodate the celiac trunk, the mesenteric arteries, etc. “Circumferential” is used herein generally to reference a direction in line with the circumference or perimeter of the affected vessel. As noted above, support for distended branching vessels may be provided by a sleeve extending from the aperture 328. The various shapes described herein may generally be combined to cover most or all the aorta and/or other blood vessels, as discussed below with respect to FIGS. 5a and 5b. Moreover, the various shapes may be combined in a single device 212 in which instance the device 212 will have preformed segments with the particular shapes discussed herein or otherwise. For example, the device 212 may be preformed to include a first curved shape segment, followed by a cylindrical segment, followed by a second curved shape segment, etc.

Referring to FIG. 5a, extravascular intervention devices 212 may be implanted around various blood vessels including, but not limited to, the ascending aorta 502, the arch of the aorta 504, the thoracic aorta 506, the abdominal aorta 510, and the iliac arteries 508. A plurality of relatively small extravascular devices 212, e.g., about 1 cm to about 2 cm in length, may be implanted in a concentrically stacked arrangement as shown with regard to the abdominal aorta 510. The stacked arrangement generally facilitates implanting the device 212 to cover a relatively large affected segment of the aorta using a minimally invasive procedure. The devices may be stacked to overlap each other (not shown), to butt up against each other (as shown in FIG. 3b), or spaced apart from each other (as shown in FIG. 5a). In certain instances, the abdominal aorta 510 and the iliac arteries 508 will both be affected, in which instance the extravascular device 212 alone or in combination with a plurality of devices 212 may have a “Y” shape or a portion thereof to cover the affected arteries.

Referring to FIG. 5b, the extravascular devices 212 may also be implanted in a non-concentrically stacked arrangement, particularly around curved sections of the aorta, such as one or more of ascending aorta 502, the arch of the aorta 504, and the thoracic aorta 506. In this instance, a plurality of devices 212 or a single device having a plurality of differently shaped segments may be implanted around the affected section to conform to the curvature of the aorta. In one embodiment, the devices are implanted around the aorta to form a curve from about 180 degrees to about 360 degrees. The degree of the curvature or any other dimension of the affected blood vessel may be based on actual measurement, e.g., with a CAT scan or other imaging modality that may be used to determine the dimensions of the affected blood vessel.

FIG. 5b schematically shows the locations for five extravascular devices or a single device with five segments 512, 514, 515, 517, and 519 where the devices may be implanted to conform to the curved sections of the aorta. To conform to the curve of the ascending aorta 502, a plurality of differently shaped devices or segments 512, 514 may be used. For instance, a first device 512 may be a curved section with a first curve radius 516 and length 526, and the second device 514 may be a curved section with a second curve radius 518 that is different than the first curve radius 516 and a length 528. The curved sections may be separated by a straight section 515 with a length 529. The arch of the aorta 504 may similarly be supported with one or more devices or segments 517, 519 having curve radii 530, 521, respectively, and a lengths 532, 523 respectively. Also shown is a sleeve 534 protruding from aperture 536 to accommodate the brachiosephalic trunk.

The shape and size of the device or devices selected for particular applications may be based on measured dimensions of the affected blood vessel. In this instance, the measured dimensions may be communicated to a manufacturer that produces at least one extravascular device 212 that conforms to or resembles the desired or measured dimensions. The extravascular device 212 may, for example, be designed based on a three-dimensional representation of the affected blood vessel so as to closely resemble the shape and size of the affected blood vessel or the pre-affected, e.g., normal, shape and size of affected blood vessel. In this instance, the extravascular device will be shaped to resemble the affected blood vessel and include at least one access opening that allows the affected blood vessel to be placed there through. A plurality of differently shaped and sized devices may also be packaged in kit form for particular blood vessels and/or particular aneurysm sizes. For example, a kit for the ascending aorta may include a number of curved sections and straight sections of various lengths and/or diameters for use with 5 cm aneurysms. A kit may also be provided with a plurality of devices that when assembled collectively resemble the desired shape or the affected shape of the blood vessel. The kit may also include devices that collectively provide support for angular measurement of about 180 degrees to nearly about 360 degrees of a curved blood vessel. Additionally, the kit may include other items therein that are useful or necessary for implantation, including surgical tools, sutures, etc.

A standard thoractomy can leave a scar approximately 10 to 15 centimeters in length and result in significant post-operative pain as well as potential complications such as bleeding, infection and air leakage. The extravascular device of the present invention avoids these problems in that it may be placed by thorascope via one or more small incisions. It is understood that the extravascular device of the present invention may be placed by any minimally invasive surgical procedure which avoids the need to isolate the aorta from the structures posterior to the aorta.

The method of implanting the extravascular intervention device 212 may vary, depending on the location of the aneurysm. Implantation, for example, may be achieved by introducing the device to a site of interest in a patient using minimally invasive laparascopic techniques, or with open-surgical intervention. With regard to laparascopic procedures, the extravascular intervention devices 212 may generally be introduced into the thoracic, e.g., between a subjects ribs, or abdominal cavity through trocars, and implanted with common laparascopic tools, e.g., by rolling or collapsing the extravascular intervention device 212 into a compact shape, and passing the device 212 through the trocar to the target vessel. The extravascular device or devices 212 are preferably implanted to provide extravascular support to a significant amount of the affected blood vessel, e.g., the affected section of the blood vessel, such as greater than about 50% to about 100%, or greater.

The extravascular intervention device 212 may be opened or expanded, and placed around the vessel having the aneurysm. The longitudinal ends may then be connected to each other to provide the compressive forces necessary to maintain and/or attain the desired shape and/or size of the aneurysm. In one embodiment, the device is made of a biocompatible and preferably MRI compatible material resilient against arterial blood flow pressure preformed into a tubular shape that is resumed when the device is placed into contact with the affected blood vessel, thereby allowing the affected blood vessel to pass the through the tubular shape. When reformed into the tubular shape, the tubular structure preferably provides at least a minimal amount of compressive force against the affected section of the blood vessel; this beneficially obviates the need for a physician to maintain the proper tubular shape while trying to connect the longitudinal ends. Self-closing or locking sutures made of a biocompatible and preferably MRI compatible material resilient against arterial blood flow pressure that, when heated, automatically form pigtails for knotting the sutures may also be used. The self-closing sutures may further be preformed to tighten the connection between the longitudinal ends upon application of heat.

The extravascular intervention device 212, upon implantation around a vessel with an aneurysm 210, may be retained in place relative to the aneurysm by the frictional forces created between the inner surfaces 214 of the extravascular intervention device 212 and the vascular wall. The extravascular intervention device 212 may also be fastened to the patient's anatomy, as with sutures or staples. Alternatively, “active” fixation may be achieved with deployable internal “barbs” or by roughening the interior surface of the surfaces contacting the exterior of the vascular structure.

The extravascular intervention device 212 according to the present invention may be constructed of a variety of biocompatible and MRI-compatible material which is resilient against arterial blood flow pressure. Alternatively, non-biocompatible materials covered with biocompatible materials may be used. By biocompatible is meant that they are acceptable for implantation in the body and any adverse bodily reaction to their presence, if any, is not so great as to outweigh the benefit of the device when employed for its intended use.

A metal mesh with the appropriate geometrical features, sinusoidal and circular, and cross patterns to provide adequate flexibility may be appropriate in certain circumstances; nitinol (a super elastic nickel titanium alloy) or other biocompatible and preferably MRI compatible material resilient against arterial blood flow pressures or stainless steel can be used. Non-resorbable polymers and elastomers such as silicones, fluoropolymers, polyolephins or polyurethanes might also be used. In addition, the subject devices can be fabricated from composites of two or more different types of materials, etc, e.g., the device may be fabricated from a blood impermeable membrane attached to a structural article or scaffold.

Those skilled in the art will recognize that certain materials are preferred in connection with certain uses of the invention. In general the material should be comprised of one or more materials which are biocompatible and non-toxic to the vessels into which they are inserted. In general the device is used for connecting vessels of the cardiovascular system and therefore should be comprised of a material which provides a high degree of hemocompatibility. The material should not prevent growth of a new intima layer. The material used in the construction of the invented device should be designed to have thickness and properties appropriate for the stiffness and flexibility of the vessel into which the device is inserted. It should be noted that artery walls continuously dilate and contract due to the systole and diastole of the heart. If the device is too rigid the device can cause irritation and injury to the intima layer of the vessel. Accordingly, the device should be designed to avoid any inflammatory response or immune response that has adverse consequences. In addition to having the desired degree of flexibility and composition the device should be designed so that it does not present protrusions or disruptions to the flow of material through the vessels which are being connected by the device. Interruption of flow can cause clots to form which could in certain circumstances be fatal to the patient.

Examples of such materials include, without limitation, graft material and shaped memory alloys (SMAs) such as synthetic polymers, metals, alloys, polyester, Dacron, Gortex, polytetrafluoroethylene, polyethelene, polypropylene, polyurethane, silicon, stainless steel, titanium, platinum, combinations thereof, etc. Alternatively, the extravascular intervention device could be made of one or more shaped memory alloys (SMAs) or a combination of graft material and SMAs. SMAs are a group of materials that demonstrate an ability to return to some previously defined shape or size when subjected to the appropriate thermal procedure. Generally, these materials can be plastically deformed and, upon exposure to thermal manipulation, will return to the pre-deformation shape. Some SMA material is considered to be two-way shaped memory alloys because they will return to the deformed shape upon proper thermal activation. SMAs include Ag—Cd alloys, Cu—Al—Ni alloys, Cu—Sn alloys, Cu—Zn alloys, Cu—Zn—Si alloys, Cu—Zn—Sn alloys, Cu—Zn—Al alloys, In—Ti alloys, Ni—Al alloys, Ni—Ti alloys, available under the trade name NITINOL, Fe—Pt alloys, Mn—Cu alloys, Fe—Mn—Si alloys, and the like.

The selection of materials for the device of the present invention is based upon the mechanical and physical properties needed for the intended use. That is, the material should be MRI “compatible.” In the context of a diagnostic or interventional procedure, this refers to the ability to accurately image the device. Preferred materials are those which overcome magnetic attraction of magnetic members, RF heating effects of conductive members, and visualization under MRI. Examples of suitable preferably MRI compatible materials, include but are not limited to, engineering resins such as polysulfone, polyethylene fiber, optical fibers, fiberglass, and carbon fiber-epoxy composites. However, it is understood that any preferably MRI compatible material may be used.

Material selected for construction is generally based on the properties thereof, e.g., the modulus of elasticity, the tensile strength, etc., in relation to the desired characteristics of the tubular structure of the device, e.g., flexibility, elasticity, etc. Biocompatible and preferably MRI compatible material resilient against arterial blood flow pressures may further be selected based on the transition temperature, i.e., the temperature at which the material returns to the preformed shape. The material selected for the extravascular intervention device 212 may be constructed into a woven fabric, mesh, or sheet, or a combination thereof, that may be formed into a tubular structure that it is capable of being implanted around the vessel with the aneurysm 210. The extravascular intervention device may also be made of a biodegradable or biosorbable material, such as PGA (Polyglycolic Acid), PLA (Polylactic acid) and co-polymers of the two, that wear away when no longer necessary or desired. For example, the device may degrade upon the completion of the desired treatment, e.g., when the aneurysm walls have been thickened, with or without a therapeutic agent, sufficient to limit or prevent further dilation of the aneurysm. The material selected should generally provide sufficient strength characteristics to resist against significant deformation when exposed to systolic blood pressure.

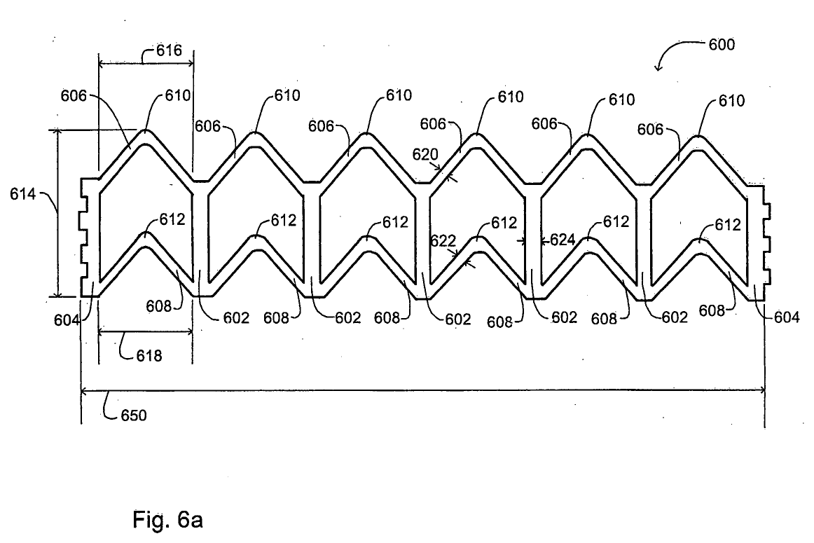

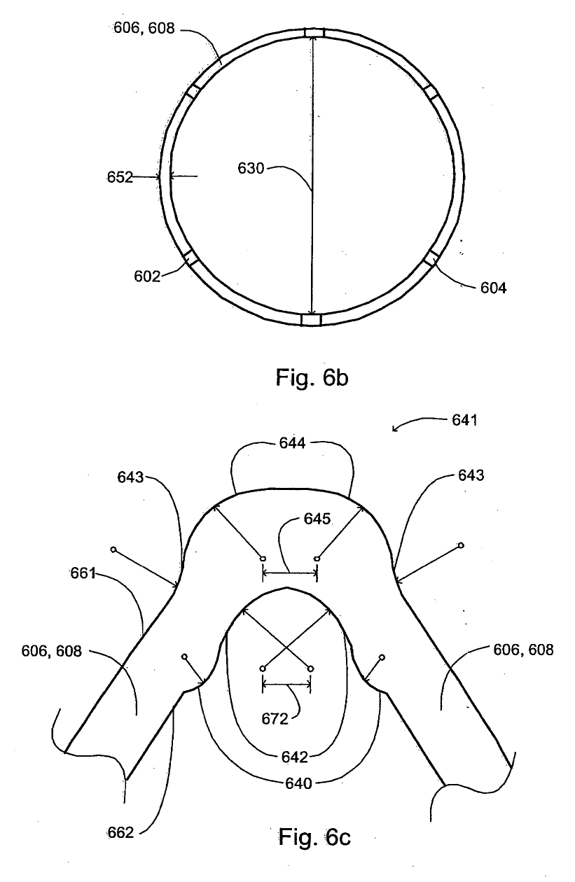

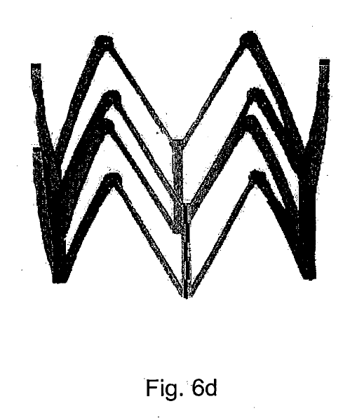

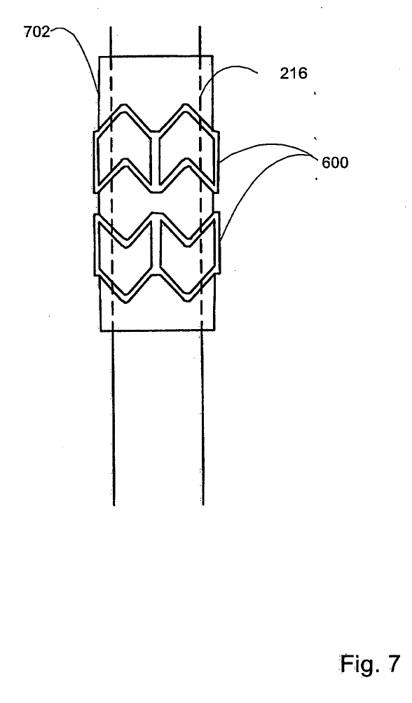

In one embodiment of the invention, a collapsible extravascular intervention device 600 is provided that may be collapsed for introduction into the target cavity, and subsequently expanded, automatically or otherwise, for implantation around the vessel with the aneurysm 210. Collapsibility may be provided in a variety of ways, such as with a device formed from a loosely woven fabric or netting. Referring to FIGS. 6a-6b, in one embodiment, collapsibility is provided with a device 600 having a plurality of longitudinal members 602, 604 connected to each other with a plurality of flexural members 606, 608. Each of the flexural members 606, 608 has a bend 610, 612 that, upon application of the requisite force, provides flexure which results in the narrowing of the lateral distances 616, 618 between the longitudinal members 602, 604, and the corresponding narrowing of the overall circumferential length 650 and diameter 630 of the device 600. The extravascular intervention device 600 correspondingly expands with or without the application of force, to increase the lateral distances 616, 618 between the longitudinal members 602, 604, thereby yielding a desired overall circumferential length 650 or diameter 630 to match the circumference and diameter of the desired shape and size of the affected vascular walls. In one embodiment, the device is made of a biocompatible and preferably MRI compatible material resilient against arterial blood flow pressure, and has a preformed tubular shape that expands when the device is placed into contact with the affected blood vessel.

The number and dimensions of the longitudinal members 602, 604 and of the flexural members may vary according to the shape and size of the affected vessel targeted for extravascular intervention and of the desired shape and size of the vessel that results from use of the extravascular device. In one embodiment, the device comprises two end longitudinal members 604 and a plurality of, e.g., five, central longitudinal members 602, each longitudinal member 602, 604 having a first end and a second end connected by flexural members to an adjacent longitudinal member's first end and second end 606, 608, respectively. It is understood that the number of central longitudinal members 602 may vary based on, e.g., the vessel diameter and the desired amount of support to be supplied thereto, including as 3 to 3000, or greater.

The thickness of the device 652 may vary depending on the properties of the material from which the device is constructed. The widths 620, 622, 624 of the longitudinal members 602, 604 and the flexural members 606, 608 generally vary depending on the properties of the materials, the desired flexibility or elasticity for collapsing and expanding the device 600, and the desired amount of support that the device provides to the aneurysm vessels. Greater support would necessarily require larger widths, thereby limiting or reducing the amount of unsupported space existing between the longitudinal and flexural members.

In one embodiment, end longitudinal members 604 include corresponding interlocking geometries that allow the longitudinal ends 604 to engage each other, thereby restricting longitudinal movement. The interlocking geometries may be provided by one or more keys disposed on one of the end longitudinal members 604 and a corresponding receiving geometry on the opposing longitudinal end member. The interlocking geometries further allow a plurality of extravascular devices to be fastened to each other, to increase the circumferential length 650. As noted above, extravascular intervention devices 600 may be longitudinally stacked to yield a desired effective longitudinal length and to accommodate vessels branching from the aneurysm.

The flexural members 606, 608 may further include at least one flexible element, as at the bends 610, 612. A flexible element generally provides additional flexibility to the flexural members, such that the flexural members 606, 608 behave elastically when the extravascular intervention device 600 is collapsed, i.e., when subjected to stresses below the device material's elastic limit. This allows the device to expand and return substantially to the original un-collapsed orientation, i.e., the expanded orientation, upon removal of the forces acting on the device 600 to maintain a collapsed orientation. Referring to FIG. 6c, the flexible element 641, according to one embodiment, is an essentially semicircular element that is defined by obtuse circumferential geometry 661 and acute circumferential geometry 662. “Obtuse” pertains to the side of the flexible element facing the obtuse angle created by the bend 610, and “acute” pertains to the side of the flexible element facing the acute angle created by the bend 610. The obtuse and acute circumferential geometries 661,662 generally include a plurality of arcs that define the semicircular flexible element 641, such that additional material is provided in areas of the flexible element 641 where localized stress concentrations may occur, as at the bend 610 or at the intersection of the longitudinal members 602, 604 and the flexural members 606, 608.

In one embodiment, the obtuse circumferential geometry 661 includes a pair of first central arcs 644 having centers that are separated by a first central distance 645. The first central arcs 644 interface with the flexural members 606, 608 by a pair of first exterior arcs 643 which are tangential to both the first central arcs 644 and the flexural members 606, 608. The acute circumferential geometry 662 includes a pair of second central arcs 642 which interface with the flexural members 606, 608 by a pair of second exterior arcs 640. The centers of the second central arcs are separated by a second central distance 672. The second exterior arcs 640 intersect tangentially with the second central arcs 642; however, they do not do so with respect to the flexural members 606, 608, thereby creating a protruding section of additional material at the second exterior arcs 640. The “center of an arc” generally relates to the origin of the radius of the arc. Additionally, “separated” is used herein to denote lateral or circumferential distance between the centers of the arcs. The distance between the centers of the arcs may be a negative number, as with the acute circumferential geometry, such that the radii of the central arcs overlap; the distance between the centers of the arcs may also be a number greater than or equal to zero, as with the obtuse circumferential geometry, such that the radii of the central arcs either coincide or do not overlap.

Implantation procedure, as noted above, may be achieved with minimally invasive endoscopic, e.g., thoracoscopic techniques, or with open-surgical intervention. With regard to endoscopic procedures, the extravascular intervention device 212, 600 is generally introduced into the target cavity through a trocar in a collapsed orientation, thereby allowing the device 212, 600 to fit within or on the trocar. Upon insertion in the target cavity, the extravascular intervention device 212, 600 is unfolded, expanded, or otherwise opened, automatically or otherwise, so that the device 212, 600 may be placed around the affected vessel in a manner forming a tubular structure. The extravascular intervention device 600 may also be implanted around the vessel, as shown in FIG. 7, after placing a base material in the form of a sheet, mesh, or fabric, such as Dacron, around the affected vessel to prevent the vessel from bulging out of the apertures created between the flexural and longitudinal members 606 608, 602. The base material may simply be placed loosely around the affected section of the vessel and held in place with the intervention device, fastened at longitudinal ends to provide additional support to the aneurysm, or connected to the intervention device.

The base material may further include, or be impregnated with, medicines or other compositions, thereby permitting the extravascular and/or intravascular administration of pharmaceuticals to the affected blood vessel. Exemplary pharmaceuticals include, without limitation, compositions comprising therapeutic agents for use in stimulating vascular growth, e.g., smooth-muscle-cell-proliferation, in order to increase the thickness of the affected blood vessel and/or to create surface scarring and retraction of the affected blood vessel, and compositions comprising any other type of therapeutic agents. A non-inclusive list of therapeutic agents includes sirolimus, everolimus, or any other related composition, paclitaxel, basic fibroblast growth factor, dexamehasone, abciximab, and other IIb/IIIa antagonists, angiopeptin, TGF, tetracycline, and other sclerosing agents, fullerenes, etc. The compositions, with or without timed-release mechanisms, may be coated directly onto at least a portion of the inner surface and/or the outer surface of the device for extravascular and/or intravascular administration of therapeutic agents, respectively. It is to be understood that the device may generally be used to administer therapeutic agents extravascularly and/or intravascularly for other purposes simply as a method for administering therapeutic agents, including, but not limited to, the inhibition of smooth-muscle-cell proliferation, etc.

The longitudinal members 604 of the extravascular device may be fixed in relation to each other with fastening means, such as sutures, wire, staples, clamps, ties, pins, snaps, Velcro, zippers, buttons, hooks, or any type of tension mechanism, glue/bonding agents, magnets, welds, etc., thereby providing the necessary compressive forces to support and relieve the stress on the affected vascular walls. Alternatively, or in addition, the extravascular intervention device may be deployed through a small incision guided by fiber-optic visualization or robotic deployment.

As noted above, endovascular graft implants have a tendency to migrate, e.g., downstream in the direction of blood flow. Implant migration is unpredictable insofar as migration cannot be attributed to any particular factors that would prompt physicians to monitor the implant for migration. As a result, physicians typically monitor implant migration for all implant patients. The extravascular device of the present invention may also be used in this respect to stabilize endovascular graft implants extravascularly. In this instance, the extravascular device is implanted around the affected blood vessel to compress the endovascular implant in order to resist or limit migration. Alternatively or in addition, the device of the present invention may be used to secure an endovascular graft (endograft) by implanting at least one extravascular segment at a level corresponding to the proximal segment of the endograft, and another exostent segment at a level corresponding to the distal end(s) of the endograft. These will limit certain endoleaks and minimize endograft migration below and/or above the graft to lock it in place or otherwise limit migration.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. An extravascular aneurysm treatment device comprising a a tubular shaped structure having a longitudinal access opening that allows an affected blood vessel to be placed through the tubular structure, wherein the device has a size that when implanted to partially encircle the affected blood vessel provides extravascular support thereto, displaces vascular walls of an affected blood vessel section inwardly, maintains resiliency under outward arterial blood pressure forces or a combination thereof.

2. The extravascular aneurysm treatment device of claim 1, wherein the device has a size that when implanted to partially encircle the affected blood vessel maintains an affected size of the affected blood vessel, displace vascular walls of the affected section inwardly, or a combination thereof.

3. The extravascular aneurysm treatment device of claim 1, wherein the device has a preformed shape selected from a group consisting of: a cylindrical shape, a conical shape, an hour-glass shape, a spherical segment shape, curved a cylindrical shape, and combinations thereof.

4. The extravascular aneurysm treatment device of claim 1, wherein the device has a preformed shape conforming to a shape of the affected blood vessel.

5. The extravascular aneurysm treatment device of claim 6, wherein the conforming shape comprises an affected shape or a pre-affected shape.

6. The extravascular aneurysm treatment device of claim 1, wherein the extravascular devices collectively have a longitudinal length to provide extravascular support to a significant amount of an affected section of the blood vessel.

7. The extravascular aneurysm treatment device of claim 1, wherein the device has a longitudinal length between about 0.5 cm and about 50 cm.

8. The extravascular aneurysm treatment device of claim 1, wherein the device has a longitudinal length between about 1 cm and about 120 cm.

9. The extravascular aneurysm treatment device of claim 1, wherein the device has an inner diameter of about 3 mm to 21 cm.

10. The extravascular aneurysm treatment device of claim 1, wherein the device is sized based on actual measurements of an affected blood vessel.

11. The extravascular aneurysm treatment device of claim 1, wherein the device has a shape based on actual measurements of an affected blood vessel.

12. The extravascular aneurysm treatment device of claim 1, wherein the device comprises at least one curved shape for providing extravascular support to a curved blood vessel.

13. The extravascular aneurysm treatment device of claim 12, wherein the extravascular device provide support for about 180 degrees to about 360 degrees of the curved blood vessel.

14. The extravascular aneurysm treatment device of claim 1, wherein the longitudinal ends comprise complimentary geometry for connecting the ends to each other.

15. The extravascular aneurysm treatment device of claim 14, wherein the longitudinal ends comprise interlocking geometry.

16. The extravascular aneurysm treatment device of claim 1, wherein the device comprises an aperture to accommodate blood vessels branching from the affected blood vessel.

17. The extravascular aneurysm treatment device of claim 16, comprising at least one sleeve sized for providing extravascular support to an affected branch vessel.

18. The extravascular aneurysm treatment device of claim 1, comprising a plurality of extravascular devices shaped to be implanted in a non-concentrically stacked arrangement.

19. The extravascular aneurysm treatment device of claim 1, wherein the extravascular devices have a curved shape for non-concentric implantation.

20. The extravascular aneurysm treatment device of claim 1, comprising a pleurality of wherein the extravascular devices have interlocking geometry at their respective ends for connecting the devices to each other in a stacked arrangement.

21. An extravascular aneurysm treatment device, the device comprising a plurality of differently shaped segments formed in a tubular structure having a longitudinal access opening that allows an affected blood vessel to be placed through the tubular structure, wherein the devices has a size and shape that when implanted to partially encircle the affected blood vessel provides extravascular support thereto, displaces vascular walls of an affected blood vessel section inwardly, maintains resiliency under outward arterial blood pressure forces or a combination thereof.

22. The extravascular aneurysm treatment device of claim 21, wherein the plurality of differently shaped segments collectively resemble a shape of the affected blood vessel.

23. The extravascular aneurysm treatment device of claim 22, wherein the differently shaped segments are based on actual measurements of the affected blood vessel.

24. The extravascular aneurysm treatment device of claim 21, wherein the device comprises at least one access opening to accommodate blood vessels branching from the affected blood vessel.

25. The extravascular aneurysm treatment device of claim 24, wherein the device comprises at least one sleeve to provide extravascular support to an affected branch blood vessel.

26. The extravascular aneurysm treatment device of claim 1, wherein the device comprises a biocompatible and MRI-compatible material wherein such material is resilient against arterial blood flow pressure.

27. A method of treating an aneurysm comprising the steps of: introducing an extravascular device to a patient's aneurysm;
communicating the device with an outer wall of the blood vessel;
positioning the device to partially encircle the outer wall of the blood vessel comprising an aneurysm; and
thereby providing extravascular support thereto, displacing vascular walls of an affected blood vessel section inwardly, maintaining resiliency under outward arterial blood pressure forces or a combination thereof.

28. A method for treating an aneurysm comprising the steps of: a) imaging an aneurysm to be treated to determine its size and topograghy; selecting an aneurysm treatment device as in claim 1 for use in treating an aneurysm; and c) implanting the aneurysm treatment device on the outer wall of the blood vessel containing the aneurysm.

29. A kit for treating an aneurysm, the kit comprising a plurality of extravascular devices formed in a tubular shaped structure each having a longitudinal access opening that allows an affected blood vessel to be placed through the tubular structure, wherein the devices collectively have a size that when implanted to partially encircle the affected blood vessel provides extravascular support thereto, displaces vascular walls of an affected blood vessel section inwardly, and maintains resiliency under outward arterial blood pressure forces.

30. An extravascular device for treating an aneurysm, said device comprising:
means for allowing an affected blood vessel to be placed through a tubular structure having a longitudinal access opening;
means for partially encircling an effected blood vessel; and optionally
means for attaching the device;
wherein the devices collectively have a size that when implanted to partially encircle the affected blood vessel provides extravascular support thereto, displaces vascular walls of an affected blood vessel section inwardly, and maintains resiliency under outward arterial blood pressure forces.

31. The extravascular aneurysm treatment device of claim 1, wherein the plurality of extravascular devices are sized to be implanted in a stacked arrangement comprising at least one of a spaced apart arrangement, a butted against each other arrangement, and an overlapped arrangement.
